(19) United States
(12) Patent Application Publication
(10) Pub. No.: US 2003/0135270 A1
(43) Pub. Date: Jul. 17, 2003

(54) Polyurethane sufrace buttressed cardiac valve suture ring

(76) Inventor: Eugene M. Breznock, Winters, CA (US)

Correspondence Address: CAMPBELL & FLORES LLP, 4370 LA JOLLA VILLAGE DRIVE, 7TH FLOOR, SAN DIEGO, CA, 92122, US

(21) Appl. No.: 10/054,696

(22) Filed: Jan. 17, 2002

(51) Int. Cl. A61F 2/24

(52) U.S. Cl. 623/2.42; 623/2.41

(57) ABSTRACT

The present invention provides a heart valve prosthesis device having an occluder, an occluder base and a suture ring, where the occluder base and suture ring have a coating of anti-thrombotic/anti-fibrotic/anti-pannus material on the surface of the occluder base to approximately one-half to two-thirds to the outer peripheral edge of the suture ring, wherein the coating prevents thrombus, fibrosis and pannus from forming on the surfaces it coats. For example, the invention provides an improved heart valve prosthesis device, where the heart valve prosthesis is a mechancical heart valve, or a bioprosthetic heart valve. In addition, the invention provides an improved heart valve prosthesis where the anti-thrombotic/anti-fibrotic/anti-pannus material is polyurethane or similar material; and the invention also provides an improved suture ring for use in combination with a heart valve prosthesis device having an occluder and an occluder base, wherein the improvement comprises a coating of anti-thrombotic/anti-fibrotic/anti-pannus material on the surface of the suture ring, where the coating extends from the inner peripheral edge of the suture ring to the outer peripheral edge of the suture ring, where the coating prevents thrombus, fibrosis and pannus from originating from the surface it coats. For example, the invention provides an improved heart valve prosthesis device, where the heart valve prosthesis is a mechancical heart valve, or a bioprosthetic heart valve. In addition, the invention provides an improved heart valve prosthesis where the anti-thrombotic/anti-fibrotic/anti-pannus material is polyurethane, or similar material.

### BACKGROUND OF THE INVENTION

1. Field of Invention

This invention relates to implantable mechanical and bioprosthetic devices, and more specifically, heart valves.

2. Background Information

The human heart is the primary organ that moves blood through the body. It circulates blood to and from the lungs for oxygenation, and then to all points of the rest of body and back to the heart. The critical function of regulating the flow of blood through the various chambers of the heart is carried out by the four heart valves, pulmonary, tricuspid, aortic and mitral. With advancing age, heart valves can begin to fail due to damage from disease or infections. Faulty heart valves can also be the result of congenital factors. Some heart valve conditions can be treated with medication. However, very often the only recourse for the patient is surgical replacement of the faulty valve.

Although replacement heart valve surgery has become commonplace, it is still a complicated and risky operation. The procedure requires the patient being placed on a heart lung machine, which oxygenates and circulates the patients blood while the surgeon performs an open heart procedure. Upon entering the heart, the surgeon evaluates the condition of the existing valve, and if necessary, removes the valve and replaces it with a prosthetic valve.

There are two types of prosthetic valves in use, mechanical and bioprosthetic. A typical mechanical prosthetic heart valve is comprised of an occluder(s), an occluder base, and a suture ring. The occluder(s) are mounted to the occluder base, and the occluder base is in turn attached to the suture ring, which is attached to the heart valve annulus in place of the defective natural valve. The occluder base is annular and provides a passageway through which the blood flows. The occluder(s) alternately open and close, thereby regulating the flow of blood. There can be one or more occluders. On the outer edge of the occluder base there is usually an external, circumferential surface typically configured as a groove to facilitate attachment of the occluder base to a suture ring. The suture ring is generally made from a knit fabric tube which is rolled into a toroidal form and which is secured to the periphery of the occluder base of the prosthesis. The suture ring is, in turn, affixed to the heart tissue.

A standard bioprosthetic heart valve bears a close resemblance, both in appearance and function, to the natural valve it is replacing. It has valve leaflets that are usually made from chemically treated animal tissue, such as the heart valves from a pig heart. The harvested valves are fixed in glutaraldehyde or similar fixatives in order to make them suitable for human implantation. Similar to the mechanical valve, the bioprosthetic valve has an occluder base to which the valve leaflets are attached, and through which the blood flows. Around the periphery of the occluder base is a suture ring used for affixing the valve to heart tissue. As is the case with a mechanical prosthetic valve, the ring is generally made of a knit fabric tube which is rolled into a toroidal form, and which is secured to the periphery of the occluder base of the prosthesis.

The overall design of prosthetic heart valves has been gradually refined over the years. However, there remain unaddressed issues relating to the suture rings. For example, suture rings are made from synthetic materials that can and often lead to thrombus, fibrosis and pannus in the replacement valve. These conditions can hinder the functioning of the replacement valve, and over time may require replacement of the prosthetic valve.

Any one or combination of the above-mentioned complications could result in the prosthetic heart valve recipient having to undergo a second procedure to remedy the condition. A second procedure means additional risks for the recipient, and potentially substantial costs to the health care provider or insurer, in addition to the cost of a replacement heart valve. Hence, it is of considerable benefit to the recipient, as well as, the health care provider and insurer, to minimize the need to undergo these second procedures. Thus there is a need for an improved prosthetic heart valve that is less susceptible to thrombus, fibrosis and pannus. The present invention satisfies this need and provides related advantages as well.

### SUMMARY OF THE INVENTION

The present invention provides a heart valve prosthesis device comprising an occluder, an occluder base and a suture ring, said occluder base and suture ring having a coating of anti-thrombotic/anti-fibrotic/anti-pannus material on the surface of said occluder base to the outer peripheral edge of the suture ring, such that said coating inhibits or prevents thrombosis, fibrosis and pannus from originating from the surfaces it coats. For example, the invention provides a heart valve prosthesis device, where the heart valve prosthesis is a mechancical heart valve, or a bioprosthetic heart valve. In addition, the invention provides an improved heart valve prosthesis where the anti-thrombotic/anti-fibrotic/anti-pannus material is polyurethane or like material.

The invention also provides an improved suture ring for use in combination with a heart valve prosthesis device comprising an occluder and an occluder base, wherein said improvement comprises a coating of anti-thrombotic/anti-fibrotic/anti-pannus material on the surface of said suture ring wherein said coating extends from the inner peripheral edge of the suture ring to the outer peripheral edge of the suture ring, such that said coating inhibits or prevents thrombus, fibrosis and pannus from originating from the surface it coats. For example, the invention provides an improved heart valve prosthesis device, where the heart valve prosthesis is a mechanical heart valve, or a bioprosthetic heart valve. In addition, the invention provides an improved heart valve prosthesis where the anti-thrombotic/anti-fibrotic/anti-pannus material is polyurethane or like material.

### BRIEF DESCRIPTION OF THE DRAWINGS

FIG. 1. Top View of a Mechanical Heart Valve This figure depicts a top view of a mechanical heart valve and its component parts, the occluder 15, occluder base 16, and suture ring 17. The figure also shows the location of the anti-thrombotic/anti-fibrotic/anti-pannus coating 1 to 2 on the atrial surface of the heart valve.

FIG. 2. Cross-sectional View of a Mechanical Heart Valve. This figure shows the orientation of the heart valve to the direction of the flow of blood. Also shown is the location of the coating 3 to 4 on the ventricular surface of the heart valve.

FIG. 3. Cross-sectional Perspective View of a Mechanical Heart Valve. This figure shows from the cross-sectional perspective view of a mechanical heart valve, and the location of the anti-thrombotic/anti-fibrotic/anti-pannus coating on the atrial and ventricular surfaces of the heart valve.

FIG. 4. Top View of a Bioprosthetic Heart Valve. This figure depicts the top view of a bioprosthetic heart valve, and its component parts, the leaf occluders 25, occluder base 26 and suture ring 27. The figure also shows the location of the anti-thrombotic/anti-fibrotic/anti-pannus coating from 21 to 22 on the atrial surface of the heart valve.

FIG. 5. Cross-sectional View of a Bioprosthetic Heart Valve. This figure depicts the orientation of the heart valve to the direction of the flow of blood.

FIG. 6. Cross-sectional Perspective View of a Bioprosthetic Heart Valve. This figure shows from a cross-sectional perspective view of a bioprosthetic heart valve, and its component parts, and the location of the anti-thrombotic/anti-fibrotic/anti-pannus coating on the atrial surface 21 to 22, and ventricular surface 23 to 24 of the heart valve.

### DETAILED DESCRIPTION OF THE INVENTION

The present invention provides a heart valve prosthesis device comprisng an occluder, an occluder base and a suture ring, the occluder base and suture ring having a coating of anti-thrombotic/anti-fibrotic/anti-pannus material on the surface of the occluder base to the outer peripheral edge of the suture ring such that the coating inhibits or prevents thrombus, fibrosis and pannus from forming on the surfaces it coats. For example, the invention provides a heart valve prosthesis device, where the heart valve prosthesis is a mechancical heart valve, or a bioprosthetic heart valve. In addition, the invention provides a heart valve prosthesis where the anti-thrombotic/anti-fibrotic/anti-pannus material is polyurethane or similar material.

The invention also provides an improved suture ring for use in combination with a heart valve prosthesis device having an occluder and an occluder base, wherein the improvement comprises a coating of anti-thrombotic/anti-fibrotic/anti-pannus material on the surface of the suture ring wherein the coating extends from the inner peripheral edge of the suture ring to half the distance to the outer peripheral edge of the suture ring such that the coating inhibits or prevents thrombus, fibrosis and pannus from forming on the surface it coats. This leaves sufficient sewing ring fabric to anchor the valve to the heart tissue. For example, the invention provides an improved heart valve prosthesis device, where the heart valve prosthesis is a mechancical heart valve, or a bioprosthetic heart valve. In addition, the invention provides an improved heart valve prosthesis where the anti-thrombotic/anti-fibrotic/anti-pannus material is polyurethane.

Terms and Definitions

As used herein, the terms “anti-thrombotic/anti-fibrotic/anti-pannus material” and “less thrombogenic/fibrotic/pannus” refers to biocompatible materials or compositions that inhibit or prevent the occurrence of thrombus.

As used herein, the term “atrial surface” refers to the surface of a heart valve prosthesis that is facing the backflow direction.

As used herein, the term “ventricular surface” refers to the surface of a heart valve prosthesis that is facing the outflow direction.

Heart valves play a critical role in the functioning of the heart. The valves permits blood to flow in one direction, moving from a first chamber to a second chamber, while preventing the blood from back flowing from the second chamber back to the first chamber. For example, the mitral valve permits blood to flow from the left atrium into the left ventricle, while preventing blood from back flowing from the left ventricle back to the left atrium, especially when the left ventricle contracts to pump the blood through the aorta to the rest of the body. As used herein, the term “outflow” refers to the direction that blood is permitted to flow through the replacement heart valve, such as blood movement from the atrium to the ventricle. In contradistinction, the term “backflow” refers to the direction that blood is prevented from flowing by the heart valve prosthesis, such as blood movement from the ventricle to the atrium. A prosthetic heart valve or heart valve prosthesis functions in place of a diseased or defective natural heart valve.

A prosthetic heart valve is made up of three primary elements, an occluder, an occluder base and a sewing ring. The occluder is an assembly which alternatively occludes and permits the flow of blood through the annulus of the heart valve in one direction only. The occluder base is toroidal shape. The occluder is attached to the occluder base and blood flows through the center opening of the occluder base. The suture ring, which is sometimes referred to as the sewing ring, is a toroidal shaped structure, which is affixed to the outer circumference of the occluder base. Suture rings are generally made from a synthetic fiber material, such as, Dacron, Teflon™, polyester, and the such. The suture ring is the element of the heart valve prosthesis that is physically affixed, most often sutured to the location in the heart where the defective natural heart valve had been removed. The prosthetic heart valve is affixed over the heart valve opening or heart valve annulus that is exposed by the removal of the defective or diseased natural heart valve. As used herein, the term “affixed” means to physically attach, join or fasten one object to another, such as, affixing the suture ring of heart valve prosthesis to heart tissue. Examples of affixing include, but are not limited to suturing, gluing (as in the use of surgical or tissue adhesives), clamping with a metal or polymer wire, or staples.

There are two basic types of replacement heart valves, mechanical and bioprosthetic. They differ primarily in the type of occluder that is utilized. Mechanical heart valves utilize mechanical occluders, such as, a ball and cage assembly, single leaflet disk valves, or bileaflet disk valves. The specific type of occluder is not critical to the functioning of the present invention. The occluder assembly is generically represented in FIG. 3 as a single leaflet disk valve 15, however, it should be recognized that any of the aforementioned types of occluders would function equally. The occluder is mounted onto the occluder base 16. The occluder base is circular in shape with a center opening that provides a passage through which the blood flows. The occluder base provides a foundation from which the occluder alternately permits and restricts the flow of blood through the opening of the occluder base. Affixed to the outer peripheral surface of the occluder base is the suture ring 17. The suture ring is toroidal in shape. The occluder base is attached to the suture ring, and the suture ring is affixed to heart tissues and allowed to heal with the formation of fibrous tissue to the heart tissue annulus such that it can function in place of the natural heart valve.

Bioprosthetic replacement heart valves utilize leaflet occluders made from animal tissue or human tissue 25, that mimic the action of the natural heart valve as shown in FIG. 6. The type of material from which the bioprosthetic heart valves are constructed is not critical to the present invention. The leaflets occluders are attached to an occluder base 26. The occluder base is circular in shape. The center opening provides a passage through which the blood flows. The occluder base provides a base to which the leaf occluders are attached from which the they alternately permit and restrict the flow of blood through the central opening of the occluder base. Affixed to the outer peripheral surface of the occluder base is the suture ring 27. The suture ring is toroidal in shape. With the occluder base attached to the suture ring, the suture ring is, in turn, affixed to heart tissues and allowed to heal with the formation of fibrous tissue to the heart tissue annulus such that it can function in place of the natural heart valve.

The design of suture rings have evolved in an effort to improve their function, to simplify the implantation procedure, and to promote the process of healing the prosthetic valve to the native annulus. However, there has been little change made in the choice of materials for suture rings. Suture rings are generally made from a synthetic polymer material, such as, Teflon™, Dacron, polyester, polypropylene, etc. A suture ring can consists of silicon or similar filler with Dacron, velour or similar material woven or stitched over the filler. The surface of currently used suture rings are coarse due to the cloth material used to cover it. This predisposes the suture ring to thrombus formation early in the post-operative period. As healing progresses, the cloth material can also contribute to severe pannus/fibrosis developing several months to a year after implantation. The consequences of pannus or fibrosis encroachment onto a prosthetic heart valve can be drastic, and potentially catastrophic. In a mechanical valve prosthesis, pannus/fibrosis can lead to inhibition of the action of the valve occluder by limiting its ability to open and close properly. In a tissue bioprosthetic valve the pannus/fibrosis usually continues from the sewing ring onto the tissue leaflets. Once on the leaflet base, the fibrosis continues and over several months to years time can fuse the leaflets at their commissure, distort individual leaflets, and/or stiffen leaflets to a point that they do not open or close completely. The end result of this overzealous healing response typically is a tissue valve that is both stenotic and insufficient.

It has been discovered that a coating of an anti-thrombotic/anti-fibrotic/anti-pannus material, such as, polyurethane, inhibits or prevents the formation of thrombus, fibrosis and pannus. The coating of polyurethane or similar materials act as a barrier by blocking the development of pannus/fibrosis, thereby preventing the undesired pannus/fibrosis response from jeopardizing the proper functioning of a prosthetic heart valve

Polyurethane is the reaction product of polyisocyanates and polyols in the presence of suitable catalysts and additives. Polyurethane can be made as a solid with varying degrees of flexibility, or as a liquid. The isocyanate monomer makes up the “hard” segment of the polymer, while the polyol portion makes up the “soft” segment. The overall physical characteristics of the polymer are determined by ratio of hard and soft segments. For example, polyurethanes with large hard segments are strong, rigid plastics; whereas, polyurethanes with large soft segments are flexible, rubbery materials. In addition the composition of the reaction composition, and reaction conditions can also influence the physical characteristics. Polyurethanes are used in many different applications in both its solid and liquid form. For example, flexible polyurethane foams are used to make bedding, sofas, cushions, carpet backs and car seats. Rigid foams are used for insulation in freezers, refrigerators, and roofs. Elastomeric polyurethanes are used for the soles of running shoes because of their durability. Polyurethane fibers are used for making spandex, which is a fabric material known for its strength and elasticity. Polyurethane coatings are used in paints, wood finishing, floor protection, and the paper, textile and leather industries. Liquid polyurethanes are found in coating applications, ranging from bridge coatings to floor sealers to tank linings.

In recent years, polyurethanes have found use medical applications. Both hard, rigid polyurethane resins and soft, elastomeric polyurethane resins are utilized in the healthcare industry. Some medical applications include light-weight casts, artificial kidney components and catheters. According to the Polyurethane Handbook by Gunter Oertel, “. . . polyurethanes appear to be the most promising class of polymers in in-vivo applications. Reasons for this are their high mechanical strength, flexibility, fatigue resistance, and tissue compatibility.” Oertel, Gunter: Polyurethane Handbook 2nd Edition. Hanser, New York 1993.

As used herein, the term “polyurethane” or “urethane” refers to such liquid polyurethanes that have suitable characteristics such that it can be applied as a coating onto medical devices, such as prosthetic heart valves, or the suture rings to such prosthetic heart valves.

A thrombus is an adherent to the vascular endothelium or endomyocardium, which is distinct from a simple blood clot. A blood clot results from the activation of the coagulation cascade and can form in vitro or in situ in the post mortem state. Fibrosis is the formation of fibrous tissue as a reparative or reactive process, as opposed to formation of fibrous tissue as a normal constituent of an organ or tissue. Pannus is the formation of a membrane of granulation tissue covering a normal surface. All three conditions are brought about by the presence of materials that are recognized by the body as not being part of the body. In extreme instances of these complications, additional surgery may be required to correct or negate the condition.

The current invention can be used with any commercially available prosthetic heart valves which employ the use of a suture ring or sewing ring device for affixing the replacement valve to the heart. Prosthetic heart valves are categorized according to whether a mechanical or bioprosthetic occluder is used. In fact, it is generally the occluder type that varies from model to model and manufacturer to manufacturer. All prosthetic valves, whether mechanical or bioprosthetic, have the common feature of a occluder base, to which the occluder is mounted, with a center opening through which the blood flows. Affixed to the periphery of the occluder base is a suture ring, which in turn is affixed to the heart tissue at the specific location where the prosthetic heart valve is to be placed. The current invention can be used to equal advantage with prosthetic mechanical heart valves having occluder mechanisms, such as, ball and cage, single leaflet disk and bileaflet disk, which include, but are not limited to the St. Jude Valve Bileaflet valve, manufactured by St. Jude Medical, Inc., One Lillehei Plaza, St. Paul, Minn. 55117; the On-X Valve, manufactured by Medical Carbon Research Institute, LLC. 8200 Cameron Rd, St A-196, Austin, Tex. 78754; the Carbomedics Valve, manufactured by Sulzer Carbomedics, Inc. 1300 East Anderson Lane, Austin, Tex. 78752; and the Edwards Duromedics Valve, manufactured by Baxter-Edwards, 17221 Red Hill Ave., Irvine, Calif. 92614. Further, the current invention can also be used to equal advantage with bioprosthetic valves, which include, but are not limited to the Carpentier-Edwards Porcine Valve, manufactured by Baxter Healthcare Corporation, Edwards CVS Division, 17221 Red Hill Ave., Irvine, Calif. 92614; the Carpentier-Edwards Pericardial Valve, manufactured by Baxter Healthcare Corporation, Edwards CVS Division, 17221 Red Hill Ave., Irvine, Calif. 92614; and the St. Jude Toronto Stentless Porcine Valve (SPV), manufactured by St. Jude Medical, Minneapolis, Minn.

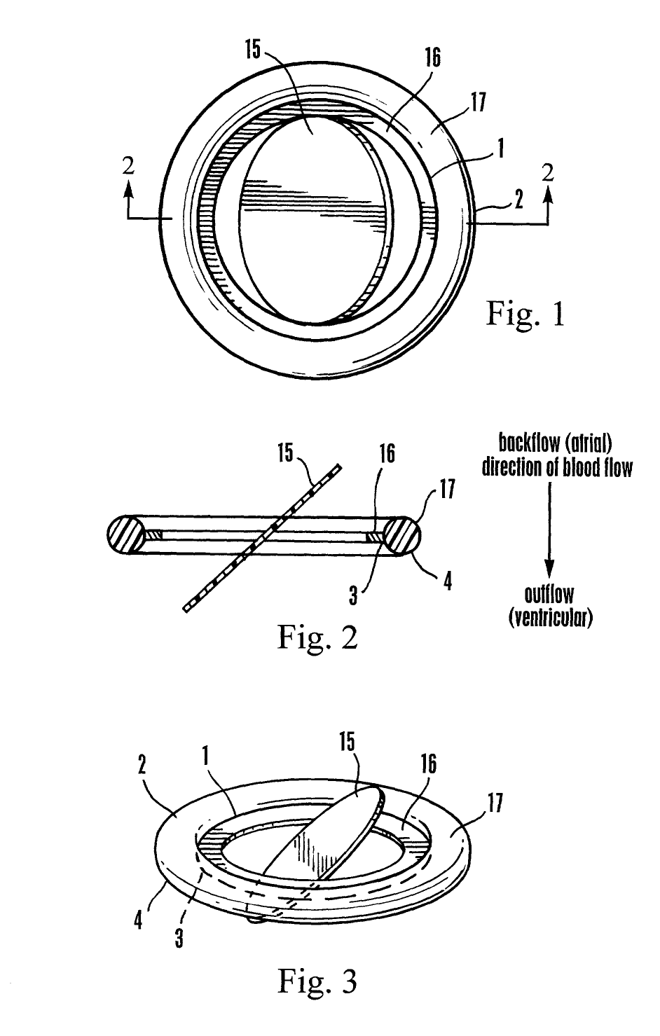

Generally for a mechanical heart valve prosthesis according to the present invention, a coating of anti-thrombotic/anti-fibrotic/anti-pannus material, such as, biocompatible polyurethane, epoxy, polypropylenes, polysulfone, Teflon™, or polyethylenes, preferably polyurethane, is applied to the prosthesis. The coating begins from about the occluder base 16 and extends distal to the occluder to about one-half to two-thirds of the peripheral edge of the suture ring 17 on atrial and ventricular surfaces of the prosthesis. As depicted in FIGS. 1, 2, and 3 the atrial and ventricular coatings extend from 1 to 2, and 3 to 4, respectively. Alternatively, a majority of the surface area of the suture ring is coated. A small portion at the outer periphery of the suture ring is not coated to allow fibroblasts to infiltrate the suture ring materials and form a strong adhesion between the suture ring and adjacent heart tissue. The thickness of the coating may vary without impairing the function of the coating, preferably the coating when dry is about 0.25 mm in thickness. The coating is applied to the atrial and ventricular surfaces of the prosthesis using a brush, or any like implements, made of suitable material for the coating material and of suitable size for the prosthesis.

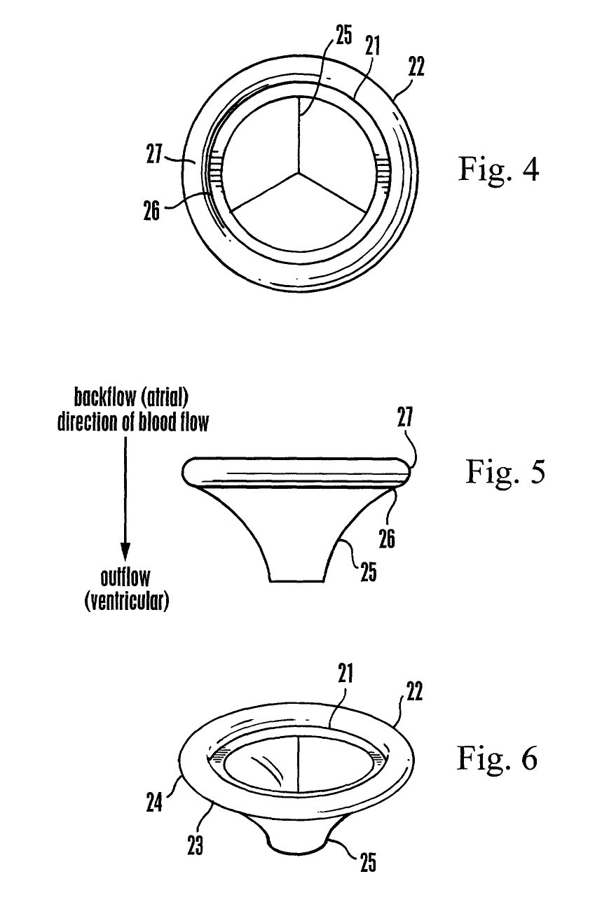

Generally for a bioprosthetic heart valve prosthesis, a coating of anti-thrombotic/anti-fibrotic/anti-pannus material, such as, polyurethane, biocompatible epoxy, polypropylenes, polysulfone, Teflon™, or polyethylenes, preferably polyurethane, is applied to the suture ring of the prosthetic heart valve. The coating begins from about the base of the leaflet valves 26 and extends to about the peripheral edge of the suture ring 27 on the atrial and ventricular surfaces of the prosthesis. As depicted in FIGS. 4, and 6, the atrial and ventricular coatings extend from 21 to 22, and 23 to 24, respectively. Alternatively, a majority of the surface area of the suture ring is coated. A small portion at the outer periphery of the suture ring is not coated to allow fibroblasts to infiltrate the suture ring materials and form a strong adhesion between the suture ring and adjacent heart tissue. The thickness of the coating may vary without impairing the function of the coating, preferably the coating when dry is about 0.25 mm in thickness. The coating is applied to the atrial and ventricular surfaces of the suture ring using a paint brush, or any like implements that are used for applying a coating of material to a surface, of suitable composition for the coating, and size for the suture ring.

Although the invention has been described with reference to the disclosed embodiments, it should be understood that various modification can be made without departing from the spirit of the invention. Accordingly, the invention is limited only by the following claims.

What is claimed is:

1. A heart valve prosthesis device comprising an occluder, an occluder base and a suture ring, said occluder base and suture ring having a coating of anti-thrombotic/anti-fibrotic/anti-pannus material on the surface of said occluder base to the outer peripheral edge of the suture ring such that said coating prevents thrombus, fibrosis, and pannus from forming on the surfaces it coats.

2. The heart valve prosthesis device of claim 1, wherein said heart valve prosthesis is a mechanical heart valve.

3. The heart valve prosthesis device of claim 1, wherein said heart valve prosthesis is a bioprosthetic heart valve.

4. The heart valve prosthesis device of claim 1, wherein said anti-thrombotic/anti-fibrotic/anti-pannus material is polyurethane.

5. An improved suture ring for use in combination with a heart valve prosthesis device comprising an occluder and an occluder base, wherein said improvement comprises a coating of anti-thrombotic/anti-fibrotic/anti-pannus material on the surface of said suture ring wherein said coating extends from the inner peripheral edge of the suture ring to a distance of approximately one-half to two-thirds to the outer peripheral edge of the suture ring such that said coating prevents thrombus, fibrosis and pannus from forming on the surfaces it coats.

6. The improved heart valve prosthesis device of claim 5, wherein said heart valve prosthesis is a mechanical heart valve.

7. The improved heart valve prosthesis device of claim 5, wherein said heart valve prosthesis is a bioprosthetic heart valve.

8. The improved heart valve prosthesis device of claim 5, wherein said anti-thrombotic/anti-fibrotic/anti-pannus material is polyurethane.

9. A method of inhibiting or preventing the formation of thrombus, fibrosis and pannus on a heart valve prosthesis device comprising an occluder, an occluder base and a suture ring, said method comprising,
applying a coating of anti-thrombotic/anti-fibrotic/anti-pannus material to the atrial surface of said heart valve prosthesis device beginning from about said occluder base and extending distal to about one-half to two-thirds of the peripheral edge of said suture ring, and
applying a second coating of anti-thrombotic/anti-fibrotic/anti-pannus material to the ventricular surface of said heart valve prothesis device beginning from about said occluder base and extending distal to about one-half to two-thirds of the peripheral edge of said suture ring.

10. The method of claim 9 wherein said anti-thrombotic/anti-fibrotic/anti-pannus material is polyurethane.

11. The method of claim 9 wherein said heart valve prosthesis device is a mechanical heart valve.

12. The method of claim 9 wherein said heart valve prosthesis device is a bioprosthetic heart valve.

13. A method of inhibiting or preventing the formation of thrombus, fibrosis and pannus on a heart valve prosthesis device comprising an occluder base, an occluder and a suture ring, said method comprising,
applying a coating of anti-thrombotic/anti-fibrotic/anti-pannus material to the atrial surface of said heart valve prosthesis device beginning from about said occluder base and extending distal onto said suture ring so that a majority of said atrial surface of said suture ring is coated, and
applying a second coating of anti-thrombotic/anti-fibrotic/anti-pannus material to the ventricular surface of said heart valve prothesis device beginning from about said occluder base and extending distal onto said suture ring so that a majority of said ventricular surface of said suture ring is coated.

14. The method of claim 13 wherein said anti-thrombotic/anti-fibrotic/anti-pannus material is polyurethane.

15. The method of claim 13 wherein said heart valve prosthesis device is a mechanical heart valve.

16. The method of claim 13 wherein said heart valve prosthesis device is a bioprosthetic heart valve.
